(19) United States
(12) Patent Application Publication
(10) Pub. No.: US 2015/0306382 A1
(43) Pub. Date: Oct. 29, 2015

(54) IMPLANTABLE MEDICAL LEADS HAVING HIGH FREQUENCY SHUNTS THAT INCLUDE A CONDUCTIVE INTERCONNECT

(71) Applicant: Medtronic, Inc., Minneapolis, MN (US)

(72) Inventors: Bushan K. Purushothaman, Blaine, MN (US); Kenneth C. Gardeski, Plymouth, MN (US); Suping Lyu, Maple Grove, MN (US); Yaling Fan, Maple Grove, MN (US); Peter M. Seiler, St. Anthony, MN (US)

(73) Assignee: Medtronic, Inc., Minneapolis, MN (US)

(21) Appl. No.: 14/263,241

(22) Filed: Apr. 28, 2014

(51) Int. Cl. A61N 1/08; A61N 1/05

(52) U.S. Cl. CPC A61N 1/08; A61N 1/05; A61N 2001/086

(57) ABSTRACT

Implantable medical leads include a conductive interconnect within a high frequency shunt that dissipates high frequency current. The conductive interconnect provides an elasticity that allows a drive shaft to rotate and translate during implantation of the lead while the conductive interconnect maintains physical contact with the drive shaft and with a shunt electrode before, during, and after the implantation. The conductive interconnect may provide a low friction that presents a smooth rotation and translation of the drive shaft to provide an acceptable tactile feedback during implantation. The conductive interconnect also provides a high electrical conductivity so that a substantial amount of high frequency current flows through the conductive interconnect to the shunt electrode. The conductive interconnect may include a polymer filler that partially penetrates into the interstitial spaces of the conductive interconnect to assist in maintaining the physical integrity of the conductive interconnect.

### TECHNICAL FIELD

Embodiments relate to implantable medical leads that have a high frequency shunt from a conductive path. More particularly, embodiments relate to high frequency shunts that include a conductive interconnect.

### BACKGROUND

Implantable medical leads are routed from a location of an implantable medical device to a stimulation and/or sensing target site. The implantable medical device is typically positioned in a location where implantation is convenient, and this location may be a significant distance from the target site. The implantable medical lead provides an electrically conductive pathway that spans the distance from the position of the implantable medical device to the target site where the conductive pathway may carry stimulation signals and/or sensed physiological signals.

Conventional implantable medical leads and systems are susceptible to high frequency electromagnetic energy being coupled onto the electrical conductors within the lead as a high frequency electrical current. This high frequency current may then exit the lead via electrodes at the target site. If the high frequency energy has a significant magnitude, then there is a risk of discomfort for the patient and damage to the tissue that is adjacent to the electrodes. While most ambient conditions do not present high frequency energy at a magnitude that causes concern, magnetic resonance imaging (MRI) scans do produce levels of high frequency electromagnetic energy that raise concerns about patient discomfort and localized tissue damage.

One approach that has been taken to alleviate the high frequency currents is to include a high frequency shunt near the electrode. The high frequency shunt provides an additional path for the high frequency electrical current to take when exiting the lead. The high frequency shunt creates an electrical pathway from the electrical conductor of the lead to an additional electrode spaced from the electrode. The electrical pathway of the shunt provides a capacitive coupling between the electrical conductor and the exterior of the lead, for instance, by creating an insulative coating on the shunt electrode. The capacitive coupling allows at least a large portion of the high frequency current to pass to the exterior via the shunt electrode while the low frequency stimulation current or sensed current does not pass through the shunt. The shunt electrode is positioned away from the tissue at the target site where the stimulation or sensing electrode is located so that any energy dissipated via the shunt electrode avoids damaging the tissue at the target site. Furthermore, the shunt electrode may be positioned in an area where there is significant blood flow, such as a chamber within the heart, which is less susceptible to temperature increases from the high frequency current than the tissue that contacts the electrode used for stimulation or sensing.

The high frequency shunt may introduce challenges for leads that use a helical electrode that is driven into the tissue at the target site by turning the helical electrode via application of torque to a connector pin that in turn transfers the torque to a coil that is connected to the pin and to a drive shaft coupled to the helical electrode. In such a lead, these elements within the lead body must rotate and translate when driving the helical electrode into the tissue. The electrical continuity between the electrical conductor and the shunt electrode of the lead may be compromised as a result of the rotation and translation of the conductor that has occurred during implantation. Furthermore, because the shunt may uses a metal-to-metal connectivity of C-clips or spring clips between the rotating and translating component and the shunt electrode, the presence of the shunt may cause frictional resistance that presents an undesirable tactile feedback during implantation. The metal-to-metal connectivity of the C-clips or spring clips may further create noise on the electrical conductor of the lead due to intermittent contact between the electrical conductor and the shunt electrode.

### SUMMARY

Embodiments address issues such as these and others by providing implantable medical leads that have high frequency shunts that include conductive interconnects to establish the electrical pathway from the electrical conductor to the shunt electrode. The conductive interconnect may be constructed so that the electrical conductor, such as a driveshaft of the helical electrode, can be rotated and translated during implantation and the electrical continuity of the high frequency shunt remains effective after implantation. Furthermore, the conductive interconnect may be constructed to provide an acceptably smooth tactile feedback during implantation. The conductive interconnect may be a continuous conductive fiber structure, such as a conductive felt. The conductive felt may be constructed of carbon. A polymer filler may at least partially penetrate the continuous conductive fiber to further assist in the continuous interconnect maintaining physical integrity during and after implantation to thereby maintain the proper physical contact and electrical continuity.

Embodiments provide an implantable medical lead that includes an insulative lead body having a proximal end and a distal end and that includes a conductive electrical connector positioned at the proximal end. The implantable medical lead further includes an electrode positioned at the distal end and a shunt electrode positioned proximal to the electrode, the shunt electrode having an insulative coating on at least one side. The implantable medical lead also includes an electrically conductive path within the lead body and interconnecting the electrical connector and the electrode and a conductive interconnect disposed between the electrically conductive path and the shunt electrode to create an electrical path from the electrically conductive path to the shunt electrode. The conductive interconnect is a continuous conductive fiber structure.

Embodiments provide an implantable medical system that includes an implantable medical device and an implantable medical lead coupled to the implantable medical device. The implantable medical lead includes an insulative lead body having a proximal end and a distal end and includes a conductive electrical connector positioned at the proximal end. The implantable medical lead also includes an electrode positioned at the distal end and a shunt electrode positioned proximal to the electrode, the shunt electrode having an insulative coating on at least one side. The implantable medical lead further includes an electrically conductive path within the lead body and interconnecting the electrical connector and the electrode and a conductive interconnect disposed between the electrically conductive path and the shunt electrode to create an electrical path from the electrically conductive path to the shunt electrode. The conductive interconnect is a continuous conductive fiber structure.

Embodiments provide a method of shunting high frequency energy from a conductive path of an implantable medical lead. The implantable medical lead has a shunt electrode positioned proximal to the electrode, the shunt electrode having an insulative coating on at least one side. The implantable medical lead also has an electrically conductive path within the lead body and interconnecting the electrical connector and the electrode and a conductive interconnect disposed between the electrically conductive path and the shunt electrode to create an electrical path from the electrically conductive path to the shunt electrode. The conductive interconnect is a continuous conductive fiber structure. The method involves conducting at least a portion of the high frequency energy from the electrically conductive path through the conductive interconnect and through the shunt electrode.

Embodiments provide an implantable medical lead that includes an insulative lead body having a proximal end and a distal end, a conductive electrical connector positioned at the proximal end, an electrode positioned at the distal end, and a shunt electrode positioned proximal to the electrode. The shunt electrode has an insulative coating on at least one side. An electrically conductive path is present within the lead body and interconnects the electrical connector and the electrode. A conductive interconnect is disposed between the electrically conductive path and the shunt electrode to create an electrical path from the electrically conductive path to the shunt electrode. The conductive interconnect provides a modulus of elasticity in a range of 0.1 MPa to 10 GPa and an electrical conductivity of at least 0.001 Siemens per centimeter.

Embodiments provide an implantable medical lead that includes an insulative lead body having a proximal end and a distal end and a conductive electrical connector positioned in proximity to the proximal end. The lead includes an electrode positioned in proximity to the distal end of the lead body and a shunt electrode positioned proximal to the electrode, the shunt electrode having an insulative coating on at least one side. The lead further includes an electrically conductive path within the lead body and interconnecting the electrical connector and the electrode, at least a portion of the electrically conductive path being rotatable relative to the lead body and the shunt electrode. The lead also includes a washer shaped conductive interconnect being continuous about a circumference and being disposed between the electrically conductive path and the shunt electrode to create an electrical path from the electrically conductive path to the shunt electrode while allowing the electrically conductive path to be rotatable, with the conductive interconnect providing an electrical conductivity of at least 0.001 Siemens per centimeter.

This summary is intended to provide an overview of the subject matter described in this disclosure. It is not intended to provide an exclusive or exhaustive explanation of the techniques as described in detail within the accompanying drawings and description below. Further details of one or more examples are set forth in the accompanying drawings and the description below. Other features, objects, and advantages will be apparent from the description and drawings, and from the statements provided below.

### DESCRIPTION OF THE DRAWINGS

FIG. 1 shows an example of an implantable medical system according to various embodiments.

FIG. 2 shows an example of a longitudinal cross-section of the distal end of the lead of the implantable medical system.

FIG. 3 shows another longitudinal cross-section of the distal end of the lead showing an example of a conductive interconnect that has a continuous conductive fiber structure.

FIG. 4 shows an example of the conductive interconnect.

### DETAILED DESCRIPTION

Embodiments provide implantable medical systems, leads, and related methods where a shunt within the implantable medical lead includes a conductive interconnect. The conductive interconnect allows electrical continuity between the electrical conductor of the lead and the shunt to be maintained after the implantable medical lead has been implanted by rotating, translating, or otherwise moving an electrical conductor within a conductive path to place the distal electrode within the tissue. Additionally, the conductive interconnect allows electrical continuity between the electrical conductor of the lead and the shunt to be maintained during flexing or other movement of the lead, e.g., caused by the beating of the heart of the patient.

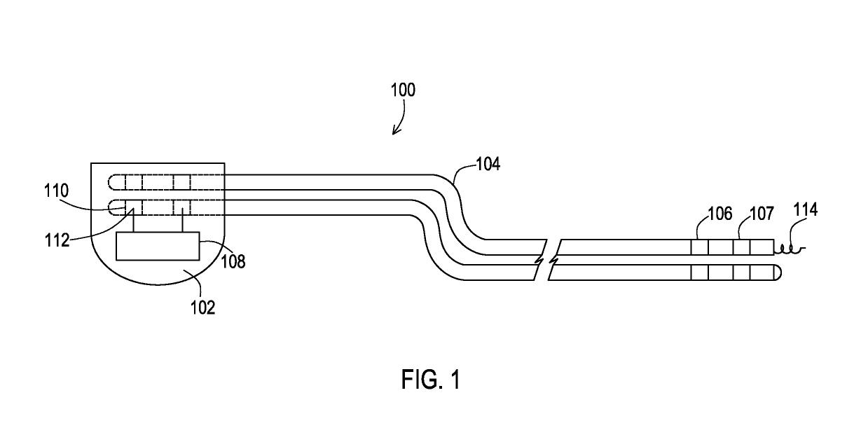

FIG. 1 shows an example of an implantable medical system 100 that includes an implantable medical lead 104 that has a shunt electrode 107. The system 100 includes an implantable medical device 102 that provides electrical stimulation and/or performs sensing of physiological signals. The implantable medical device 102 includes circuitry 108 that provides the stimulation and/or sensing function. The circuitry 108 is electrically coupled via electrical connectors 112 to proximal electrical contacts 110 of one or more leads 104 having a distal electrode 114 that may serve as a cathode when used for stimulation. The proximal contacts 110 are located in proximity to the proximal end of the lead 104 while the electrode 114 is located in proximity to the distal end. At least one of the leads 104 includes a helical electrode 114 that is implanted into tissue by rotation and translation of the electrode 114. The lead 104 may also include another distal electrode 106 for providing an anode for bi-polar stimulation and/or for sensing.

One or more of the leads 104 may also include other electrode designs including a hemispherical electrode as shown or even ring electrodes. Such a lead 104 may also include a shunt electrode 107 with an interconnect providing electrical continuity between the shunt electrode 107 and the electrical conductor within the lead 104. While such a lead may not call for rotation of a helical electrode, the conductive interconnect that corresponds to embodiments discussed below may be present to simplify construction, for instance, by avoiding interconnect welds.

Additionally, while the example of FIG. 1 shows a shunt electrode 107 that may be connected to the electrical conductor leading to the electrode 114 serving as the cathode, an additional shunt electrode may be provided that is connected to the electrical conductor lead to the electrode 106 that serves as the anode. In such a case, a conductive interconnect may be included even though the electrical conductor to the anode is not rotated during implantation as the conductive interconnect may simplify construction in this example of a shunt electrode.

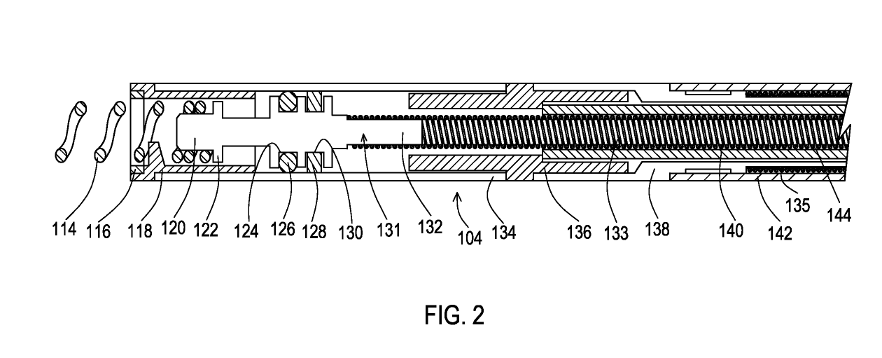

FIG. 2 shows an example of a distal end of the lead 104 shown in longitudinal cross-section. The lead 104 includes an insulative layer 142 that extends from the proximal end toward the distal end as shown. The lead 104 includes other layers includes an inner layer 140 that defines a lumen 144. The lumen 144 allows an electrical conductor 133, shown as a coil in this example, to pass from the proximal end toward the distal end. The lumen 144 also provides a passageway for a stylet to steer the lead during implantation and to also allow the coil 133 to transfer the rotation and translation motion to a proximal end 132 of a drive shaft 131. The drive shaft 131 includes a distal end 120 with a flange 122 that receives the proximal end of the helical electrode 114. Thus, rotation of the drive shaft 131 rotates the helical electrode 114. As the helical electrode 114 rotates against a fixed tooth in the distal end of the lead 104, the electrode 114 and the drive shaft 131 translate distally to allow the electrode 114 to auger into the body tissue.

The lead 104 includes an electrode 138 which is an example of the electrode 106 of FIG. 1. This electrode 138 may be connected to a separate conductor 135 that extends to the proximal end of the lead 104 to provide the complete pathway for bi-polar stimulation pulses. An insulative layer 136 is distal of the electrode 138 and separates the electrode 138 from a shunt electrode 134 which is an example of the shunt electrode 107 of FIG. 1.

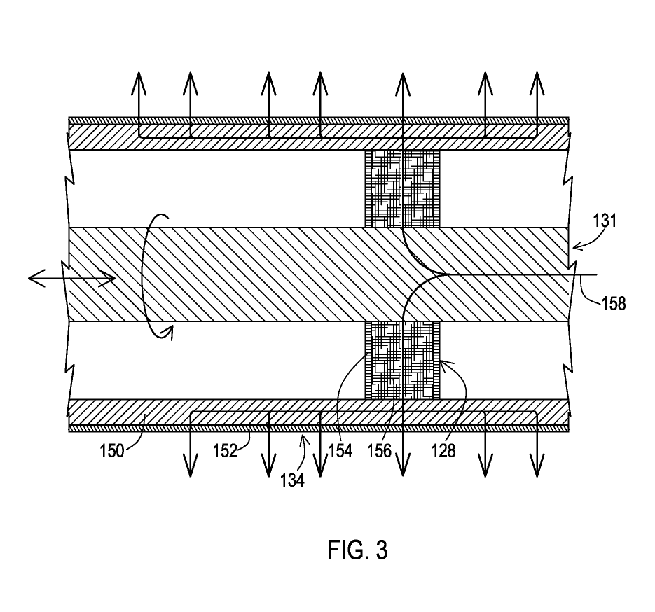

The shunt electrode 134 provides the interface to the tissue for the high frequency shunt. The shunt electrode 134 includes an insulative coating discussed in more detail below with reference to FIG. 3 in order to establish the capacitive pathway to the tissue. The shunt electrode 134 of this example surrounds the drive shaft 131 such that the electrical pathway of the high frequency shunt is created between the drive shaft 131 and the shunt electrode 134 by including a conductive interconnect 128.

The conductive interconnect 128 is positioned about the drive shaft 131 and directly between the drive shaft 131 and the shunt electrode 134. The conductive interconnect 128 physically contacts both the shunt electrode 134 and the drive shaft 131 to establish electrical continuity between them. In this example, the drive shaft 131 includes an annular groove 130 that retains the interconnect 128. When the drive shaft 131 is rotated by rotation of the coil 133, which ultimately translates the drive shaft 131 and the helix electrode 114, the conductive interconnect 128 maintains physical contact with the drive shaft 131 as the drive shaft rotates relative to the interconnect 128. The conductive interconnect 128 also maintains physical contact with the shunt electrode 134 as the conductive interconnect 128 translates relative to the shunt electrode. The conductive interconnect 128 maintains this physical contact regardless of whether the interconnect 128 rotates together with the drive shaft 131 or remains stationary with the shunt electrode 134 as the drive shaft 131 rotates. Therefore, upon the conclusion of the rotation and translation movements, the conductive interconnect 128 maintains physical integrity and conductivity while maintaining physical contact and electrical continuity between the drive shaft 131 and the shunt electrode 134.

In this example, the conductive interconnect 128 is not required to be a fluid seal. Instead, a gasket 126 such as a rubber O-ring is provided about the drive shaft 131 to provide a fluid seal between the drive shaft 131 and the shunt electrode 134. The fluid seal prevents bodily fluids that enter the distal tip of the lead 104 from entering the lumen 144 and interfering with the electrical characteristics of the conductive pathway within the lead 104.

The lead 104 includes additional layers on the distal end to further support the drive shaft 131 and helical electrode 114. An insulative distal layer 118 houses the distal end 120 of the drive shaft 131 and the proximal end of the helical electrode 114. In this example, a distal ring 116 is also included in this example to further support the helical electrode 114. This ring 116 may be a monolithic controlled release device (MCRD) which is a porous silicone ring loaded with dexamethasone acetate or dexamethasone sodium phosphate steroids. The ring 116 acts as a steroid elution device, and the steroid manages inflammation that occurs due to the injury incurred when screwing a helix electrode 114 into a myocardium.

FIG. 3 shows a cross-section of an embodiment of the distal end of the lead 104 to further illustrate the shunt. The shunt electrode 134 of this example includes a conductive layer 150 that may be a material such as a biocompatible metal like Titanium, Tungsten, Platinum, Platinum/Iridium, and MP35N. A biocompatible non-conductive coating 152 is provided on the exterior of the conductive layer 150 in this example to create a direct current (DC) open circuit which prevents any appreciable amount of the stimulation pulses or sensed physiological signals of a relatively low frequency (e.g., kilohertz range and lower) from exiting through the shunt electrode 134. In other examples, the coating 152 may be on an interior of the shunt electrode 134. However, the non-conductive coating 152 has a thickness and dielectric value that provides a capacitive coupling for high frequency current in the frequency range of interest. For example, to dissipate current in the Megahertz range from radio frequency energy produced by an MRI scan, a non-conductive material such as parylene, polyurethane, SI polyimide, or poly ether ether ketone (PEEK) may be used as the coating 152. As a specific example, this parylene coating 152 may have a thickness ranging from 0.1 micrometers to 100 micrometers for a shunt electrode 134 surface area of 49 square millimeters in order to shunt high frequency current at least ranging from 64-128 MHz.

FIG. 3 also illustrates an example of the conductive interconnect 128 that is a continuous conductive fiber structure. The structure provided by the continuous conductive fiber can be seen in section 156. Some examples of a continuous conductive fiber include carbon felt, metal fiber felt such as grade 316L stainless steel, and tantalum coated carbon fiber. The continuous conductive fibers may be randomly distributed and oriented or may be a specific distribution and orientation. Also, the continuous nature may be provided by individual conductive strands of fiber that extend from the drive shaft 131 to the shunt electrode 134 or may be provided by multiple strands of fiber that are in physical contact so as to extend from the drive shaft 131 to the shunt electrode 134. The continuous conductive fiber 156 of the conductive interconnect 128 provides a structure with physical integrity but with a low modulus of elasticity (e.g., ranging from 0.1 MPa to 10 GPa, ranging from 1 MPa to 1 GPA, or ranging from 1 MPa to 50 MPa) and a relatively high electrical conductivity (e.g., at least 0.001 Siemens per centimeter).

The conductive interconnect 128 may be formed of other structures as well and still provide the elasticity, low friction, and high electrical conductivity. For example, the conductive interconnect 128 may be constructed as tantalum foam or open cell gold foam.

The elasticity of the of the conductive interconnect 128, and particularly the continuous conductive fiber structure 156, allows the interconnect 128 to maintain physical contact with the drive shaft 131 and the shunt electrode 134 at all times, including before, during, and after the rotational and translational movements during implantation of the lead 104, and during flexing of the lead 104 due to contractions of the heart. This ensures that adequate electrical continuity is being maintained. The porosity of the continuous conductive fiber structure 156 contributes to the degree of elasticity that is achieved.

The relatively low friction of the conductive interconnect 128, and particularly the continuous conductive fiber structure 156, prevents the drive shaft 131 from being overly restricted during implantation. The relatively low friction also promotes a smooth tactile feedback for the surgeon who is applying torque to the coil 133 to cause the drive shaft 131 to rotate and translate.

The relatively high electrical conductivity of the conductive interconnect 128, and particularly the continuous conductive fiber structure 156, ensures that the high frequency current experiences a low shunt impedance. As the continuous conductive fiber structure includes paths of high conductivity throughout, the impedance of the interconnect 128 remains low over a broad range of frequencies including the typical MRI frequencies of 64 MHz and 128 Mhz. This ensures that a substantial amount of the high frequency current is dissipated through the shunt electrode 134 which decreases the amount of high frequency current that exits through the electrode 114.

The conductive interconnect 128 of this example also includes a polymer filler 154 that has partially penetrated the continuous fiber structure 156 to fill the interstitial spaces present near the outer surface of the continuous conductive fiber structure 156. Some examples of the polymer filler 154 include silicone medical adhesive (e.g., MED 1137 by NuSil Technology LLC) epoxy (e.g. Hysol, E-20HP, Henkel), liquid silicone rubber (LSR) including 30A, 50A 70A etc., Dow Corning Q74850, Q7 4735, Q7-4765 where the LSR materials can also be diluted with solvents such as heptane or xylene to achieve the appropriate viscosity for the desired conductive fiber structure penetration. Other examples include polyurethanes such as: Estane 58237, Elasthane™ 80A, Elasthane™ 55 D and Elasthane™ 75 D, and the polyurethanes can be diluted with solvents such as dimethyl acetamide (DMAC), and tetrahydrofuran (THF) to ensure the appropriate penetration of the conductive felt. Other examples also include SI polyimide: where the polyamic acid is a precursor to polyimide (Imitec/NASA Genymer SI polyimide). The polyamic acid can be diluted with solvents to achieve appropriate viscosities for application. The conductive fiber structure can be dipped in polyamic solution and then cured at sufficiently high temperatures to convert to polyimide polymer. Other examples further include polyether sulfone such as Ultrason® E 2020 P that is soluble in N-methylpyyrolidone and N, N-dimethylacetamide.

In addition to the polymer filler 154 discussed above, an adhesive may be highly diluted by mixing with a solvent such as heptane to provide complete penetration. This diluted adhesive provides bonding of the fibers for increased structural integrity, rather than filling the interstitial space, while retaining the desired electrical characteristics, namely high conductivity with a low impedance over a broad frequency range including the MRI RF frequencies of interest. Table A shows examples of heptane solvent to MED 1137 adhesive (by NuSil Technology LLC) ratio for a given size carbon felt conductive interconnect and the resulting electrical characteristics. The carbon felt of these examples is stock number 43199, 99.0%, available from Alfa Aesar (Ward Hill, Mass.).

TABLE A
Carbon Felt Impregnated With Diluted Silicon Adhesive

| | Cross- | | | | |
|---|---|---|---|---|---|
| | Sectional | | | | |
| Heptane | Area of | Thickness of | | | |
| Solvent to | Carbon Felt | Carbon Felt | | Resis- | |
| MED | Conductive | Conductive | Measured | tivity | Conduc- |
| 1137 | Interconnect | Interconnect | Resistance | (ohms/ | tivity |
| Ratio | (cm²) | (cm) | (ohms) | cm) | (S/cm) |
| 3 | 4.94 | 0.3175 | 0.9 | 14.0 | 0.071 |
| 2 | 3.6 | 0.3175 | 0.86 | 9.8 | 0.103 |
| 1 | 5.2 | 0.3175 | 2.11 | 34.6 | 0.029 |

When the polymer filler 154 partially penetrates the continuous conductive fiber structure 156, the filler 154 provides additional structural integrity for the continuous conductive fiber structure 156 and constrains the fibers to a specific locality within the lead 104 while not decreasing the elasticity and electrical conductivity below acceptable levels. The polymer filler 156 may additionally provide some degree of fluid sealing, depending upon how thoroughly the polymer filler 154 has penetrated the interstitial spaces at the radial and circumferential surfaces of the fiber structure 156.

FIG. 3 additionally illustrates the dissipation of high frequency current that may be coupled onto the electrically conductive path of the lead 104. A substantial amount of the high frequency current that is coupled onto the conductive path of the lead 104 which includes the drive shaft 131 follows the path 158. The large portion of the high frequency current enters the drive shaft 131 and then passes through the conductive fiber structure 156 into the shunt electrode 134, and capacitively couples across the non-conductive layer 152 and into the surrounding tissue and/or blood stream over the length of the shunt electrode 134.

The annular groove is not shown in FIG. 3. To assist in maintaining the position of the conductive interconnect 128 relative to the drive shaft 131, C-clips may instead be placed on either or both sides of the conductive interconnect 128. As the continuous conductive fiber already provides a highly conductive path from the drive shaft 131 to the shunt electrode 134, the C-clips are not needed for conduction and may be constructed of a non-conductive polymer such as PEEK which eliminates any electrical noise that might otherwise be caused by conductive C-clips.

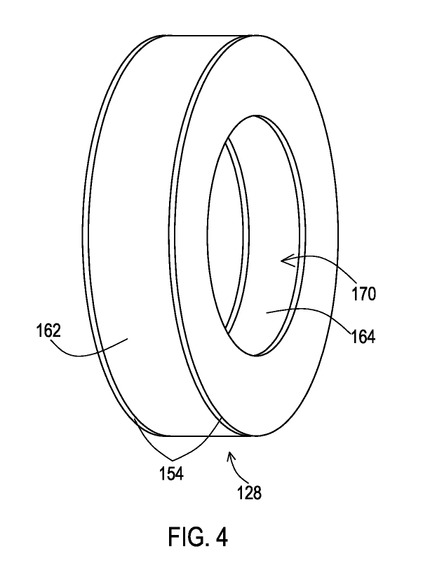

FIG. 4 shows a perspective view of the conductive interconnect 128. From this view, the polymer filler 154 that has partially penetrated into the interstitial spaces of the continuous conductive fibers 156 can be seen for both the outside circumference and for the inside circumference visible through an aperture 170. The outside circumference has a region 162 between the filler layers 154 where the continuous conductive fibers are exposed so that they make direct physical contact with the shunt electrode 134. The inside circumference has a region 164 between the filler layers 154 where the continuous conductive fibers are exposed so that they make direct physical contact with the drive shaft 131 as the drive shaft 131 passes through the aperture 170. Therefore electrical continuity is established from the drive shaft 131 to the inside circumference region 164, through the interconnect 128, and from the outside diameter region 162 to the shunt electrode 134.

FIG. 4 also illustrates that this example of an interconnect 128 is washer shaped so as to be continuous about the inner and outer circumferences where regions 162 and 164 are present. This continuity about the inner and outer circumferences provides contact around the circumference of the drive shaft 131 and also about the inner circumference of the shunt electrode 134 to further minimize the impedance at high frequencies while allowing the drive shaft 131 to rotate relative to the shunt electrode 134.

While embodiments have been particularly shown and described, it will be understood by those skilled in the art that various other changes in the form and details may be made therein without departing from the spirit and scope of the invention.

What is claimed is:

1. An implantable medical lead, comprising:
an insulative lead body having a proximal end and a distal end;
a conductive electrical connector positioned in proximity to the proximal end;
an electrode positioned in proximity to the distal end of the lead body;
a shunt electrode positioned on the lead body proximal to the electrode, the shunt electrode having an insulative coating on at least one side;
an electrically conductive path within the lead body and interconnecting the electrical connector and the electrode;
a conductive interconnect disposed between the electrically conductive path and the shunt electrode to create an electrical path from the electrically conductive path to the shunt electrode, the conductive interconnect being a continuous conductive fiber structure.

2. The implantable medical lead of claim 1, wherein the continuous conductive fiber structure is conductive felt.

3. The implantable medical lead of claim 2, wherein the conductive felt comprises carbon.

4. The implantable medical lead of claim 1, further comprising a polymer filler that at least partially penetrates into interstitial spaces of the continuous conductive fiber structure.

5. The implantable medical lead of claim 4, wherein the polymer filler is a silicone medical adhesive.

6. The implantable medical lead of claim 1, wherein the electrically conductive path includes a drive shaft connected to a helix exposed from the insulative lead body, with the conductive interconnect being positioned about the drive shaft.

7. The implantable medical lead of claim 6, further comprising an annular groove about the drive shaft with the conductive interconnect disposed within the annular groove.

8. The implantable medical lead of claim 7, further comprising a second annular groove about the drive shaft with a rubber O-ring disposed within the second annular groove.

9. An implantable medical system, comprising:
an implantable medical device;
an implantable medical lead coupled to the implantable medical device, the implantable medical lead comprising:
an insulative lead body having a proximal end and a distal end;
a conductive electrical connector positioned in proximity to the proximal end;
an electrode positioned in proximity to the distal end of the lead body;
a shunt electrode positioned on the lead body proximal to the electrode, the shunt electrode having an insulative coating on at least one side;
an electrically conductive path within the lead body and interconnecting the electrical connector and the electrode;
a conductive interconnect disposed between the electrically conductive path and the shunt electrode to create an electrical path from the electrically conductive path to the shunt electrode, the conductive interconnect being a continuous conductive fiber structure.

10. The implantable medical system of claim 9, wherein the continuous conductive fiber structure is conductive felt.

11. The implantable medical system of claim 10, wherein the conductive felt comprises carbon.

12. The implantable medical system of claim 9, further comprising a polymer filler that at least partially penetrates into interstitial spaces of the continuous conductive fiber structure.

13. The implantable medical system of claim 12, wherein the polymer filler is a silicone medical adhesive.

14. The implantable medical system of claim 9, wherein the electrically conductive path includes a drive shaft connected to a helix exposed from the insulative lead body, with the conductive interconnect being positioned about the drive shaft.

15. The implantable medical system of claim 14, further comprising an annular groove about the drive shaft with the conductive interconnect disposed within the annular groove.

16. The implantable medical system of claim 15, further comprising a second annular groove about the drive shaft with a rubber O-ring disposed within the second annular groove.

17. A method of shunting high frequency energy from a conductive path of an implantable medical lead, wherein the implantable medical lead comprises:
an insulative lead body having a proximal end and a distal end;
a conductive electrical connector positioned in proximity to the proximal end;
an electrode positioned in proximity to the distal end of the lead body;
a shunt electrode positioned on the lead body proximal to the electrode, the shunt electrode having an insulative coating on at least one side;
an electrically conductive path within the lead body and interconnecting the electrical connector and the electrode;
a conductive interconnect disposed between the electrically conductive path and the shunt electrode to create an electrical path from the electrically conductive path to the shunt electrode, the conductive interconnect being a continuous conductive fiber structure,
the method comprising conducting at least a portion of the high frequency energy from the electrically conductive path through the conductive interconnect and through the shunt electrode.

18. The method of claim 17, wherein the continuous conductive fiber structure is conductive felt.

19. The method of claim 18, wherein the conductive felt comprises carbon.

20. The method of claim 17, further comprising a polymer filler that at least partially penetrates into interstitial spaces of the continuous conductive fiber structure.

21. An implantable medical lead, comprising:
an insulative lead body having a proximal end and a distal end;
a conductive electrical connector positioned in proximity to the proximal end;
an electrode positioned in proximity to the distal end of the lead body;
a shunt electrode positioned on the lead body proximal to the electrode, the shunt electrode having an insulative coating on at least one side;
an electrically conductive path within the lead body and interconnecting the electrical connector and the electrode;
a conductive interconnect disposed between the electrically conductive path and the shunt electrode to create an electrical path from the electrically conductive path to the shunt electrode, the conductive interconnect providing a modulus of elasticity in a range of 0.1 MPa to 10 GPa and an electrical conductivity of at least 0.001 Siemens per centimeter.

22. The implantable medical lead of claim 21, wherein the conductive interconnect is a continuous conductive fiber structure

23. The implantable medical lead of claim 22, wherein the continuous conductive fiber structure is a conductive felt.

24. The implantable medical lead of claim 23, wherein the conductive felt comprises carbon.

25. The implantable medical lead of claim 22, further comprising a polymer filler that at least partially penetrates into interstitial spaces of the continuous conductive fiber structure.

26. The implantable medical lead of claim 25, wherein the polymer filler is a silicone medical adhesive.

27. The implantable medical lead of claim 21, wherein the electrically conductive path includes a drive shaft connected to a helix exposed from the insulative lead body, with the conductive interconnect being positioned about the drive shaft.

28. The implantable medical lead of claim 27, further comprising an annular groove about the drive shaft with the conductive interconnect disposed within the annular groove.

29. The implantable medical lead of claim 28, further comprising a second annular groove about the drive shaft with a rubber O-ring disposed within the second annular groove.

30. An implantable medical lead, comprising:
an insulative lead body having a proximal end and a distal end;
a conductive electrical connector positioned in proximity to the proximal end;
an electrode positioned in proximity to the distal end of the lead body;
a shunt electrode positioned proximal to the electrode, the shunt electrode having an insulative coating on at least one side;
an electrically conductive path within the lead body and interconnecting the electrical connector and the electrode, at least a portion of the electrically conductive path being rotatable relative to the lead body and the shunt electrode;
a washer shaped conductive interconnect being continuous about a circumference and being disposed between the electrically conductive path and the shunt electrode to create an electrical path from the electrically conductive path to the shunt electrode while allowing the electrically conductive path to be rotatable, the conductive interconnect providing an electrical conductivity of at least 0.001 Siemens per centimeter.
